(19) United States
(12) Patent Application Publication
(10) Pub. No.: US 2002/0098198 A1
(43) Pub. Date: Jul. 25, 2002

(54) Oral drug delivery system

(75) Inventors: Peter Watts, Nottingham (GB); Ian Lafferty, Leicestershire (GB)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: West Pharmaceutical Services Drug Delivery & Clinical Research Centre, Ltd.

(21) Appl. No.: 09/943,691

(22) Filed: Aug. 31, 2001

Related U.S. Application Data
(63) Continuation of application No. PCT/GB00/00664, filed on Feb. 24, 2000.

(30) Foreign Application Priority Data
Mar. 2, 1999 (GB) 9904629.4

(51) Int. Cl. A61K 38/00; A61K 38/39; A61K 31/717; A61K 31/7016

(52) U.S. Cl. 424/185.1; 514/21; 514/53

(57) ABSTRACT

An oral drug delivery composition comprises a solid foam forned from a protein. The foam dissolves rapidly in the mouth. The foam contains a therapeutic agent, such as a drug, an antigen or a vaccine. The foam may be prepared by a heating, freeze-drying or vacuum drying step.

### CROSS-REFERENCE TO RELATED APPLICATION

This application is a continuation of International Application No. PCT/GB00/00664, filed Feb. 24, 2000, the disclosure of which is incorporated herein by reference, which was published in the English language on Sep. 8, 2000 under International Publication No. WO 00/51593.

### BACKGROUND OF THE INVENTION

The present invention relates generally to the oral administration of drugs using a delivery system in the form of a solid foam that dissolves rapidly in the mouth, has excellent mouth-feel and is suitable for taste masking. More specifically, the present invention relates to an oral delivery composition comprising a therapeutic agent and a solid foam formed from a protein.

The administration of drugs via the mouth (oral administration) using solid dosage forms such as tablets and capsules remains the most popular means of dosing drugs. However, in certain situations, such as the treatment of children, the elderly or where a rapid onset of action is required, the use of dosage forms that dissolve rapidly in the mouth can be advantageous.

Different formulations and formulation methods have been developed to accelerate the disintegration and dissolution rate of conventional tablet systems. These have included polyethylene glycol blends, freeze-dried products and fast dissolving excipients.

Fast dissolving dosage forms for oral delivery have been reviewed by Rathbone et al. Oral Mucosal DrugDelivery, Chapter 11, Ed. Rathbone, Dekker, New York (1996).

A variety of fast dissolving oral products has been described in the prior art. Freeze-dried systems in the form of lyophilized tablets (Lyocs™) were first reported in 1978. A well known example is described in GB-A-1,548,022. This system comprises a network of the active ingredient and a water soluble or water dispersible carrier. The network is obtained by sublimating a solvent from a composition in the solid state. Another freeze-dried system is Zydis™ which is available from Scherer DDS, Swindon, UK. ZydisT has been reviewed by Seager, J Pharm., Pharmac, 50:375 (1998).

Other examples of formulations that are intended to dissolve rapidly in the mouth can be found in the prior art. U.S. Pat. No. 4,855,326 describes a melt spinnable carrier, such as a sugar, which is combined with an active ingredient and the resulting mixture spun into a “candy-floss” preparation. The spun “candy-floss” product is then compressed into a rapidly dispersing, highly porous solid dosage form.

U.S. Pat. No. 5,120,549 describes a fast-dispersing matrix system. The system is prepared by first solidifying a matrix-forming system dispersed in a first solvent and subsequently contacting the solidified matrix with a second solvent that is substantially miscible with the first solvent at a temperature lower than the solidification point of the first solvent, the matrix-forming elements and active ingredient being substantially insoluble in the second solvent, whereby the first solvent is substantially removed resulting in a fast-dispersing matrix.

U.S. Pat. No. 5,178,878 describes tablets comprising microparticles and an effervescent disintegrating agent. The microparticles contain an active pharmaceutical ingredient which is encompassed by a protective coating. On contact with saliva, the effervescent agent results in rapid disintegration of the tablet and release of the microparticles.

U.S. Pat. No. 5,298,261 describes fast-dispersing dosage forms which comprise a partially collapsed matrix network that has been vacuum-dried above the collapse temperature of the matrix. However, the matrix is preferably at least partially dried below the equilibrium freezing point of the matrix.

U.S. Pat. No. 5,587,180 describes a particulate support matrix for a tablet and method for making same, which disintegrates or dissolves in just a few seconds once placed in the oral cavity. The particulate support matrix comprises a first polymeric component which may be a polypeptide, a second polymeric component which may be a different polypeptide, and may be a hydrolyzed gelatin, and a bulking agent.

U.S. Pat. No. 5,609,883 describes the manufacture of a fast dissolving tablet using standard machinery. These tablets comprise 50% or greater of carbohydrate and alcohol as a lubricant.

WO 94/11438 describes fast-dispersing dosage forms of very low density formed by gelling, with agar, aqueous systems containing the matrix-forming elements and active ingredient, and then removing water by forced air, vacuum drying, or other drying systems.

JP-A-9216816 describes a highly water soluble solid, fast dissolving tablet produced by kneading lactilcol with water and compressing.

JP-A-9071523 describes tablets with rapid disintegration in the oral cavity. These are prepared with active, crystalline cellulose, hydroxypropyl cellulose and a lubricant. Crystalline cellulose and hydroxypropyl cellulose are used in a ratio of 1:2.

EP-A-481,294 describes a rapid dissolving tablet containing a high concentration (50% w/w) of cysteine derivatives, cellulose derivatives and sugars.

EP-A-711,547 describes tablets for rapid dissolution in the mouth. These are prepared by direct compression of an uncured matrix together with an enhancer or binder and a controlled release system.

EP-A-553,777 describes fast dissolving tablets prepared by compression molding of an active ingredient, a carbohydrate and enough water or water alcohol to wet the carbohydrate.

EP-A-590,963 describes the preparation of tablets by filling a mold with a wet paste and molding the paste under compression.

WO 91/04747 describes an effervescent dosage form comprising an effervescent agent for rapid disintegration and a plurality of microcapsules, said microcapsules including at least one systemically distributable pharmaceutical ingredient and an encapsulant substantially surrounding the pharmaceutical ingredient.

WO 96/02237 describes instant dissolution solid pharmaceuticals which comprise an active material coated with a water-dispersible binder, a cellulose expanding agent, a water soluble polyol and a diluent.

WO 97/38679 describes a fast disintegrating solid oral dosage form comprising an active substance, a filler, and a binder. The dosage forms are prepared by making a suspension or solution of the ingredients, filling into a mold and removing the solvents without freeze drying.

Foams have not been widely used for the admninistration of drugs. Rectal foams for the delivery of steroids for the treatment of colonic disease are known. Sciarra, Modern Pharmaceutics, 3rd Ed., editors—Banker and Rhodes, Dekker, New York (1996) describes quick-breaking liquid foams and mentions that it is possible to formulate edible foams to disperse cough remedies, calcium supplements, antacids, vitamins and other similar products. Sciarra also suggests that these systems may be readily acceptable to children and the geriatric population.

U.S. Pat. No. 5,079,018 describes a fast-dispersing dosage form which comprises a porous skeletal structure of a water soluble, hydratable gel or foam forming material that has been hydrated with water and rigidified in the hydrated state with a rigidifying agent. The foam forming material can be gelatin, albumin or lecithin and is rigidified with a mono- or polysaccharide. The dosage form can be formulated as wafers, tablets, granules and powders. The dehydration process is performed using a liquid organic solvent at a temperature of about 0° C. or below. Ethanol is a preferred organic solvent.

Dickenson, An Introduction to Food Colloids, p.25, Oxford University Press, Oxford (1992) has reviewed the preparation and stabilization of food colloids. Stable foams are known to be difficult to produce because bubbles are susceptible to fast drainage and rupture. Moreover, diffusion of gas from small bubbles into big bubbles can proceed quickly in the absence of a stabilizing film of a polymeric material. Stability can be provided by an insoluble adsorbed layer of a coagulated protein such as egg-white or by converting a liquid foam into a solid foam through, for example, heat treatment.

Egg-white is known to be an effective foaming agent in foods. This effect arises from the different constituents in egg-white that are important in stabilizing a liquid foam as well as the conversion of the liquid foam into a solid foam during heating. The major component of egg-white is ovalbumin, which is an effective foam stabilizer. However, the presence of highly surface active globulins can provide foam with small bubbles and a smooth texture Dickenson, supra, p.135). Ovomucoid is particularly useful in this regard. Lysosyme is another component of egg-white which can increase film strength and enhance foam stability.

Dickenson has stated that co-operative protein-protein interactions between basic protein (e.g. lysosyme) and acidic egg-white proteins are largely electrostatic. Hence, in foam stabilization the interaction of a cationic polymer with an anionic polymer can be used to form an interfacial complex. For example, two proteins of opposite charge will provide a means of enhancing foam stability. Examples are beta-lactoglobulin and bovine serum albumin. In producing a solid foam for pharmaceutical use, sugar can be added. Sucrose, fructose, glucose, mannitol, sorbitol can all be employed.

### BRIEF SUMMARY OF THE INVENTION

The present invention provides an oral delivery composition comprising a therapeutic agent and a solid foam formed from a protein. Typically, the oral delivery composition of the invention is a rapidly dissolving composition.

By “rapidly dissolving composition” we mean a composition having a weight of from 0.1 gram to 10 gram that will dissolve in the mouth in the presence of saliva in less than 300 seconds. It is preferred that the composition will dissolve in the mouth in less than 150 seconds and it is especially preferred that the composition will dissolve in less than 60 seconds.

The composition of the invention dissolves rapidly in the mouth to release the therapeutic agent.

Albumins are foam forming proteins which are suitable for use in the present invention. A preferred albumin is egg albumin. Ovalbumin or egg-white is particularly preferred.

The therapeutic agent may be a drug, an antigen or a vaccine.

### DETAILED DESCRIPTION OF THE INVENTION

Drugs suitable for use in the present invention include, but are not limited to, drugs acting on the central nervous system, drugs acting on the gastrointestinal tract, drugs acting on the cardiovascular system, antibiotic drugs, vitamins, vaccines, nutrients, drugs for analgesia, drugs for erectile dysfunction, hormones such as insulin, calcitonin, parathyroid hormone, nicotine for smoking cessation, antitussive agents, local anaesthetics, antiemetics, anticonvulsants, sedatives, and sleep induction agents.

Drugs that are preferred for use in the present invention include paracetarnol, ibuprofen, nicotine, piroxicam, enalapril, apomorphine, codeine, buprenorphine and combinations of such drugs. An especially preferred drug is paracetamol.

Antigens suitable for use in the present invention include, but are not limited to, allergen antigens, tetanus toxoid, polio myelitis, haemodulius influenzae.

The amount of therapeutic agent present in the compositions of the invention is not especially limited and will depend on several factors which will be readily apparent to the person of ordinary skill in the art, such as the nature and intended purpose of the therapeutic agent. The dose of the therapeutic agent is typically from 0.1% w/w to 90% w/w (as measured in the dry foam). The therapeutic agent is generally present in an amount of at least 1% w/w, for example 1% w/w to 80% w/w. A preferred dose of the therapeutic agent is from 2.5% w/w to 75% w/w and an especially preferred dose of the therapeutic agent is from 5% w/w to 70% w/w, particularly 5% w/w to 50% w/w.

The compositions may also include a polysaccharide. Polysaccharides stabilize the foam, enhance volume development and improve handling.

Polysaccharides suitable for use in the compositions of the invention include sucrose, for example powdered sucrose (icing sugar) or castor sugar (both available from Tate and Lyle), mannitol, sorbitol, lactose, fructose and xylitol (Sigma). Another suitable polysaccharide is carboxymethyl cellulose (CMC) which has a high viscosity and a high degree of substitution.

If the compositions of the invention contain a polysaccharide the protein and polysaccharide are together typically present in an amount of from 10% w/w to 99.9% W/W (as measured in the dry foam), generally less than 99% w/w, for example 20% w/w to 99% w/w. A preferred amount of protein and polysaccharide is 25% w/w to 97.5% w/w, an especially preferred amount is 30% w/w to 95% w/w, particularly 35% w/w to 50% w/w.

When the compositions of the invention contain a polysaccharide the ratio of protein to polysaccharide is typically from 1:1 to 1:1 0, preferably from 1:4 to 1: 8.

Of course, if the compositions do not contain a polysaccharide, the protein may represent a greater proportion of the total weight of the compositions. In this case, the amount of protein may be from 1% w/w to 99.9% w/w, generally from 1% w/w to 90% w/w (as measured in the final dried foam). A preferred amount of protein is from 15% w/w to 80% w/w and an especially preferred amount of protein is from 20 to 50% w/w.

The compositions may also include a non-ionic surfactant. Non-ionic surfactants effect the structure of the foam stabilizing layer. The effect will depend on the composition of the film, but could be an increase in foam volume or an increase in foam density. Non-ionic surfactants suitable for use in the present invention include polysorbates (commonly known as “Tweens”, ICI Chemicals).

The compositions may also include other pharmaceutically acceptable ingredients such as sweeteners, flavoring agents, taste masking agent for drugs that have a bitter taste. A suitable taste masking agent is Eudragit E100® (Registered Trade Mark of Rohm Pharma, Darmstradt, Germany). The inclusion of sugars such as sucrose will also help mask any bitter taste. The compositions may also contain pharmaceutically acceptable colorants.

Suitable sweeteners include saccharin (Sigma) and aspartame. Suitable flavorings include orange, lemon, raspberry and peppermint.

Components such as sweeteners and flavorings, if present, are typically present in the formulations of the invention in an amount of from 0.1 to 1% by weight each.

The compositions of the invention can be prepared by incorporating the therapeutic agent into the foam before the foam is solidified. Suitable solidifying methods include heat treatment, freeze drying and vacuum drying.

The compositions of the invention may be prepared by first whisking the protein, for example egg-white or ovalbumin, using a food mixer or similar equipment until a stiff foam has been produced. The therapeutic agent is typically mixed with other excipients such as sugars, artificial sweeteners, and flavoring agents. This powder is gently mixed (folded) into the foam. The therapeutic agent can also be taste masked by dissolving a taste masker, for example Eudragit E100200 , in a suitable solvent and adding this solution dropwise to the powder containing the therapeutic agent and granulating the mixture. Suitable solvents for the taste masker include dichloromethane, a water/ethanol mixture and an acetone/isopropanol mixture. After drying the granules these can be mixed with the foam. Typically, the mixture is then distributed into molds and dried. Suitable drying methods include heating in an oven (which may be done at atmospheric pressure or under reduced pressure), microwaving or freeze drying.

Alternatively, the therapeutic agent and any other excipients such as sugars, artificial sweeteners, flavoring agents and a taste masker can be mixed with the protein, for example egg-white or ovalbumin, and then the mixture whisked using a food mixer or similar equipment to produce a stiff foam. If a taste masker is used it is typically added to the therapeutic agent and other excipients as described above.

The foams can be molded or further modified by known pharmaceutical processes such as grinding and compression.

When producing a foam from egg-white, the pH may be reduced towards the isoelectric points of acidic egg-white proteins. Suitable agents for the adjustment of pH include acetic acid, citric acid, tartaric acid, succinic acid and potassium acid tartrate.

The present invention is now illustrated but not limited by reference to the following Examples.

### EXAMPLE 1

### General Method For the Preparation of a Solid Foam Product

Egg-white or reconstituted dried egg-white (ovalbumin) (obtained from Sigma, Poole, UK and Cake Art Ltd, Somerset, UK, respectively) was mixed with water using a food mixer on medium speed until a stiff foam (meringue) was formed. The drug was blended with other excipients such as various carbohydrates (sugars), sweeteners and flavoring agents using mortar and pestle and then gently mixed (folded) into the foam using a spatula. Portions (approximately 1 g) of the drug-containing foam were then filled into small molds (5 ml weighing boats) and placed in an oven (Mexcel General Purpose Oven) overnight at 60° C. (temperatures of 40 to 80° C. can also be used) to produce a solid foam.

A typical formulation is as follows:—

| | |
|---|---|
| Dried egg-white | 7.5 g (approximately equal to one egg-white) |
| Water | 35 ml |
| “Sugar” | 45 g (30 to 60 g were used) |
| Drug (if paracetamol) | up to 20 g (5 to 20 g were used) |
| Flavoring | 0.5 g |
| Sweetener | 0.5 g (both flavoring and sweetener could |
| | be included in greater or smaller amounts) |

Formulations containing orange, lemon raspberry and peppermint flavorings were prepared.

Formulations containing saccharin and aspartame as sweeteners were prepared.

### EXAMPLE 2

### A Solid Foam Containing Paracetamol Produced Using Sucrose in the Form of Castor Sugar (Formulation A)

10 g of paracetamnol and 55 g of castor sugar was slowly folded into an egg-white foam as described in Example 1. A solid foam was prepared as described in Example 1.

### EXAMPLE 3

### A Solid Foam Containing Paracetamol Produced Using Sucrose in the Form of Icing Sugar (Formulation B)

10 g of paracetamol and 55 g of icing sugar was slowly folded into an egg-white foam as described in Example 1. A solid foam was prepared as described in Example 1.

### EXAMPLE 4

### A Solid Foam Containing Paracetamol and Orange Flavor (Fornulation C)

10 g of paracetamol, 55 g of icing sugar and 0.25 g of orange flavoring were mixed in a mortar and pestle. This was then folded into one beaten egg-white and weighed into small tablet sized portions and converted into a solid foam by treating portions as described in Example 1.

### EXAMPLE 5

### A Solid Foam Containing Paracetamol, Flavor and Sweetening Agent (Formulation D)

10 g of paracetamol, 45 g of icing sugar, 0.5 g orange flavoring, 0.5 g saccharin were mixed in a mortar and pestle. This was then folded into one beaten egg-white and then processed as in Example 4.

### EXAMPLE 6

### A Solid Foam With a Peppermint Flavor (Formulation E)

A solid foam was produced as in Example 5, but 0.75 g of peppermint oil was used instead of 0.5 g orange flavor.

### EXAMPLE 7

### A Solid Foam with a Peppermint Flavor (Formulation F)

A solid foam was prepared as described in Example 6, but 1.25 g of peppermint flavor was used.

### EXAMPLE 8

### A Solid Foam Prepared Using Mannitol (Formulation G)

A solid foam was prepared as in Example 6, but mannitol was used instead of icing sugar.

### EXAMPLE 9

### A Solid Foam Prepared Using Mannitol (Formulation H)

A formulation as described in Example 8 was prepared but with 1.25 g of peppermint flavor.

### EXAMPLE 10

### Preparation of Solid Foams Using Freeze Drying

Foams as described in Examples 1 to 9 were prepared using a freeze drying process. Freeze drying was performed by freezing the foam meringues in an -80° C freezer for approximately 4 hours. The foams were then transferred to an Edwards bench top freeze-drier and dried overnight.

### EXAMPLE 11

### Preparation of Solid Foams Using Vacuum Drying

Foams as described in Examples 1-9 were prepared using a vacuum drying process.

The vacuum drying was performed using a Virtis Genesis freeze-drier (without engaging the freezer). Samples were placed in the drier at 35° C and the vacuum set to a pressure of 300 Pa.

### EXAMPLE 12

### Solid Foam With the Addition of a Polymer to Provide Taste Masking

Paracetamol 20 g, icing sugar 55 g, orange flavoring 0.5 g were mixed together in a mortar and pestle. 10 grams Eudragit E100® was dissolved in a mixture of4 g water and 66 g ethanol was added dropwise to the paracetamol blend with constant mixing (spatula) until a satisfactory granulation was achieved. The granules were wet screened (1.4 mm sieve) dried at 40° C. for 4 hours in an oven and then blended with the foam prepared as described above in Example 1. Portions (approximately 1 g) were dried in an oven and in the freezer-drier as previously described to produce solid foams. A reduction in aftertaste was achieved when evaluated in a group of volunteers.

### EXAMPLE 13

### Further Solid Foams Containing Paracetamol (Formulations L, J and K)

As in Example 1 using, dried egg-white, 15 g reconstituted with water 75 ml, to form the foam. Paracetamol 20 g, icing sugar 55 g and approximately 1 ml of orange (formulation I), lemon (formulation J) and peppermint liquid flavorings (formulation K), were mixed (mortar and pestle) and gradually added to the foam. The mixture was dried in an oven at 60° C. overnight.

### EXAMPLE 14

### Use of Eudragit E100® as a Binder to Aid Taste Masking (Formulations L1 and L2)

A solution containing Eudragit E100® 30 g in dichloromethane 100 ml was prepared.

Paracetamol 10 g, icing sugar 10 g, orange flavoring 0.1 g and aspartame 0.2 g were mixed together (mortar and pestle) and “granulated” with 6.7 ml of the Eudragit solution (=2 g of polymer). The mixture rapidly dried in air and was passed through a 0.5 mm sieve.

The granules 13.6 g, icing sugar 16 g orange flavoring 0.09 g and aspartame 0.1 g were blended together using a Turbula mixer for 5 minutes.

Dried egg-white 3 g was reconstituted with 15 ml of water and whisked until a stiff foam had formed. The powder blend was then gradually added to the meringue.

Portions were placed in molds (weighing boats) and dried in an oven at 60° C. overnight (formulation L1).

Portions were frozen (−80° C.) and dried in freeze drier overnight (formulation L2).

### EXAMPLE 15

### Use of Eudragit E100® as a Binder (Formulations M1 and M2)

These formulations were made as for formulation L but using 3.3 ml of Eudragit/dichloromethane solution (1 g of polymer). Oven dried formulations were labelled M1 and freeze dried formulations were labelled M2.

### EXAMPLE 16

### Use of Beta-Cyclodextrin for Taste Masking (Formulation N)

3 g dried egg white was reconstituted with 15 ml of water and whisked into a stiff foam as in Example 1. Paracetamol 6 g, Beta-cyclodextrin (Sigma) 5 g, icing-sugar 17 g, a lemon flavoring 0.15 g, aspartame 0.22 g were dry mixed in a Turbula mixer for 5 minutes. The powder blend was then incorporated into the foam. 2 g portions were placed in the molds (weighing boats) and the meringue dried in an oven at 40° C overnight.

### EXAMPLE 17

### Flavored Product (Formulation 0)

Approximately 2 ml of liquid lemon flavor was added to formulation J of Example 13 (post manufacture) and allowed to dry in air for 1 hour.

### EXAMPLE 18

### Use of Xylitol in Foam Preparation (Formulation P)

The foam was prepared as before as in Example 1. To one quarter portion (equivalent to 3.75 g dried egg-white and 18.75 ml water) xylitol 13 g, icing sugar 13 g, paracetamol 10 g and aspartame 0.5 g were gradually added. The product was dried in over at 60° C. overnight.

### EXAMPLE 19

### Use of Xylitol in Foam Preparation (Formulation Q)

The foam was prepared as for formulation P (Example 18) but 26 g of xylitol was added and the icing sugar was removed.

### EXAMPLE 20

### Evaluation of Solid Foams by Taste Testing in Volunteers

The properties of paracetamol containing solid foams as described in formulations A to 0 were evaluated in a group of volunteers (n=6). The time for the formulation to dissolve (melt) in the mouth and the taste and aftertaste were recorded. Details are given in Table 1. The taste and aftertaste were ranked according to a scale from 1 to 5.

TABLE 1
Evaluation of solid foams containing paracetamol

| | Melt in mouth | | |
|---|---|---|---|
| Formulation# | time (sec) | Taste* | After taste |
| A | 10 | 4 good | 3 slight |
| B | 15/20 | 3 OK | 2 bitter |
| C | 20/30 | 2 chewy | 3 slight |
| D | 10 | 2 gritty/OK | 3 slight |
| E | 20 | 2 gritty/OK | 3 slight |
| F | 20 | 2 gritty/minty/OK | 3 slight |
| G | 15 | 4 good melt | 4 very slight |
| H | 15 | 4 good melt | 4 very slight |
| I | 20 | 3 good melt | 3 slight |
| J | 20 | 4 good melt | 4 very slight |
| K | 20 | 4 good melt | 4 very slight |
| L1 | 20 | 4 good melt | 4 very slight |
| L2 | 20 | 4 good melt | 4 very slight |
| M1 | 20 | 4 good melt | 2 bitter |
| M2 | 20 | 4 good melt | 2 bitter |
| N | 15 | 4 good melt | 4 very slight |
| O | 20 | 4 good melt | 4 very slight |
| P | 10/15 | 5 good melt | 4 very slight |
| Q | 10/15 | 3 chewy | 4 very slight |

#Formulation Key

| | |
|---|---|
| A | Castor sugar or sucrose/paracetamol |
| B | Icing sugar/paracetamol |
| C | Icing sugar/paracetamol/orange |
| D | Granulation/paracetamol/orange |
| E | Granulation/paracetamol × 2/peppermint |
| F | Granulation/paracetamol × 2/extra peppermint |
| G | Mannitol granulation/paracetamol × 2/peppermint |
| H | Mannitol granulation/paracetamol × 2/extra peppermint |
| I | Icing sugar/paracetamol/orange solution |
| J | Icing sugar/paracetamol/lemon solution |
| K | Icing sugar/paracetamol/peppermint solution |
| L | Icing sugar/paracetamol/orange/Eudragit E100 ® (2 g) |
| M | Icing sugar/paracetamol/orange/Eudragit E100 ® (1 g) |
| N | Icing sugar/paracetamol/lemon/Beta-cyclodextrin/aspartame |
| O | Icing sugar/paracetamol/lemon solution/post manufacture lemon |
| | solution |
| P | Icing sugar/xylitol/paracetamol/aspartame |
| Q | Xylitol/paracetamol/aspartame |

*Taste Key

| | |
|---|---|
| 1 | Very poor |
| 2 | Poor |
| 3 | Average |
| 4 | Good |
| 5 | Excellent |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An oral delivery composition comprising a therapeutic agent and a solid foam formed from a protein and which is prepared by heating.

2. A composition according to claim 1, wherein the protein is an albumin.

3. A composition according to claim 2, wherein the albumin is egg-albumin.

4. A composition according to claim 3, wherein the albumin is ovalbumin or egg-white.

5. A composition according to claim 1, which further comprises a polysaccharide.

6. A composition according to claim 5, wherein the polysaccharide is selected from the group consisting of sucrose, powdered sucrose (icing sugar), castor sugar, mannitol, sorbitol, lactose, friuctose, xylitol, and carboxymethyl cellulose (CMC).

7. A composition according to claim 1, which dissolves in the mouth in less than 60 seconds.

8. A composition according to claim 1, which further comprise a flavoring agent.

9. A composition according to claim 1, which further comprises a taste masking agent.

10. A composition according to claim 1, wherein the therapeutic agent is selected from the group consisting of a drug, an antigen and a vaccine.

11. A composition according to claim 10, wherein the drug is paracetamol.

12. A composition according to claim 10, wherein the drug is selected from the group consisting of codeine, ibuprofen, piroxicam, enalapril, apomorphine, nicotine, buprenorphine, and combinations thereof.

13. A method for the preparation of a composition according to claim 1, comprising a heating, freeze-drying or vacuum drying step.

14. A method of oral delivery of a therapeutic agent comprising preparing a solid foam from a protein by heating and incorporating the therapeutic agent in the foam.

15. The method according to claim 14, wherein the therapeutic agent is selected from the group consisting of a drug, an antigen and a vaccine.

16. A composition comprising a therapeutic agent and a solid foam formed from a protein and which is prepared by heating for use as a medicament.
